(19) United States
(12) Patent Application Publication
(10) Pub. No.: US 2009/0197291 A1
(43) Pub. Date: Aug. 6, 2009

(54) Assays Using Nanoparticles

(76) Inventors: Yuri Volkov, County Dublin (IE); Yury Rakovich, County Dublin (IE); Louri Kuzmich Gounko, County Dublin (IE); John Donegan, County Kildare (IE); Dermot Kelleher, County Dublin (IE); Siobhan Mitchell, Dublin (IE)

Correspondence Address: JACOBSON HOLMAN PLLC, 400 SEVENTH STREET N.W., SUITE 600, WASHINGTON, DC, 20004, US

(21) Appl. No.: 12/084,239

(22) PCT Filed: Oct. 27, 2006
(86) PCT No.: PCT/IE2006/000123

§ 371 Date: Nov. 4, 2008

Related U.S. Application Data
(60) Provisional application No. 60/730,365, filed on Oct. 27, 2005.

(30) Foreign Application Priority Data
Oct. 27, 2005 (IE) 2005/0721

(51) Int. Cl. C12Q 1/68; G01N 33/68; G01N 21/64; G01R 27/08

(52) U.S. Cl. 435/29; 436/86; 436/87; 436/172; 324/692

(57) ABSTRACT

A method for quantitatively and qualitatively determining the presence of a macromolecule comprises providing nanoparticles in a buffered solution, adding a test sample to the buffered nanoparticle solution, and measuring the difference between the buffered nanoparticles in the presence and absence of the test sample. The nanoparticles are preferably less than 100 nm in size.

The invention relates to the use of nanoparticles in detection and quantification assays.

### BACKGROUND

Recent interdisciplinary technological developments have led scientists to embrace nanoparticle methodology for biomedical applications (Bruchez et al., 1998; Chan et al., 1998; Akerman et al., 2002). Of a wide variety of nanoparticles available, quantum dots (QDs) in particular, or colloidal semiconductor nanocrystals are robust particles of size and composition tunable emission. They exhibit wide absorption profiles allowing excitation of various QDs simultaneously, narrow emission spectra and excellent photo stability (Mattoussi et al., 2002; Michalet et al., 2001; Chan et al., 2002), making them potentially readily traceable in the cells and tissues of the living organisms.

Initial hurdles of biocompatibility, solvent-based production, complex surface chemistry and low quantum yield have now been overcome allowing investigation of nanoparticle activity in biological systems (Chan et al., 1998; Bruchez et al., 1998). These advances include capping CdSe particles with ZnS to allow for an increased quantum yield (Chan et al., 1998), while Peng and colleagues have utilised alternative precursor materials to generate large quantities of high quality nanocrystals (Peng et al., 2001).

QDs display dimensional similarities to biomolecules permitting their bioconjuagtion and use as sensors. To date QD studies have been performed primarily using CdSe particles. Early attempts at labelling cells included adding transferrin-QD bioconjugates to HeLa cells thereby allowing receptor-mediated endocytosis (Chan et al., 1998). Also, the avidin-biotin system was employed to label F actin filaments where biotinylated CdSe nanocrystals were used to label fibroblasts incubated in phalloidin-biotin and streptavidin (Bruchez et al., 1998). CdSe—CdS nanocrystals coated with trimethoxysilylpropyl urea and acetate were found to bind with high affinity in the cell nucleus (Bruchez et al., 1998). CdSe QDs have also been used in metastatic assessment as markers for phagokinetic tracks (Parak et al., 2002). The first reports of in vivo use show QD-peptide conjugates targeting tumor vasculature (Akerman et al., 2002). Later studies using ZnS coated CdSe QDs encapsulated in PEG micelles show DNA binding and successful microinjection into Xenopus embryos (Dubertret et al., 2002).

Detection and selective functional modification of complex cell surface receptor repertoire, intracellular components and individual biomolecules in cell systems and in vitro applications constitute a priority task in modern biology and medicine. The most typical examples are drug screening, flow cytometry, cell imaging, protein and DNA detection. Traditional methods for detecting biological compounds in vivo and in vitro rely mostly on the use of radioactive markers. For example, these methods commonly use radioactive-labelled probes such as nucleic acids labelled with ³²P or ³⁵S and proteins labelled with ³⁵S or ¹²⁵I to detect biological molecules. These labels are effective because of the high degree of sensitivity for the detection of radioactivity. However, many basic difficulties exist with the use of radioisotopes. Such problems include the need for specially trained personnel, general safety issues when working with radioactivity, inherently short half-lives with many commonly used isotopes, and disposal problems due to full landfills and governmental regulations. As a result, current efforts have shifted to utilising non-radioactive methods of detecting biological compounds. These methods often consist of the use of fluorescent molecules as tags (e.g. fluorescein, ethidium, methyl coumarin, rhodamine, etc.), or the use of chemiluminescence as a method of detection. Fluorescence is the emission of light resulting from the absorption of radiation at one wavelength (excitation) followed by nearly immediate re-radiation usually at a different wavelength (emission). Fluorescent dyes are frequently used as tags in biological systems. For example, compounds such as ethidium bromide, propidium iodide, Hoechst dyes (e.g. benzoxanthene yellow) interact with DNA and fluoresce to visualize DNA. Other biological components can be visualized by fluorescence using such techniques as immunofluorescent microscopy, which utilizes antibodies labelled with a fluorescent tag and recognizing particular cellular target. For example, in a commonly used two-step immunodetection method, “secondary” polyclonal (rabbit- or goat-anti-mouse) antibodies tagged with fluorescein or rhodamine enable one to visualize “primary” monoclonal antibodies (typically raised in mice or respective hybridoma cells) bound to specific cellular targets. However, simultaneous use of several “primary” urine monoclonal antibodies to detect multiple targets is limited by the species specificity of the “secondary” fluorescently-tagged reagents leading in this case to severe cross-reactivity and false positive staining results. In one aspect the invention is directed to providing a solution to this problem.

Fluorescent dyes also have applications in non-cellular biological systems. For example, the advent of fluorescently-labelled nucleotides has facilitated the development of new methods of high-throughput DNA sequencing and DNA fragment analysis (ABI system; Perkin-Elmer, Norwalk, Conn.). Despite certain progress, there are a number of chemical and physical limitations to the use of organic fluorescent dyes. One of these limitations is the variation of excitation wavelengths of different coloured dyes. As a result, simultaneously using two or more fluorescent tags with different excitation wavelengths requires multiple excitation light sources. This requirement thus adds to the cost and complexity of methods utilising multiple fluorescent dyes. Another drawback when using organic dyes is the deterioration of fluorescence intensity upon prolonged exposure to excitation light. This fading is called photobleaching and is dependent on the intensity of the excitation light and the duration of the illumination. In addition, conversion of the dye into a nonfluorescent species is irreversible. Furthermore, the degradation products of dyes are organic compounds, which may interfere with biological processes being examined. Another drawback of organic dyes is the spectral overlap that exists from one dye to another. This is due in part to the relatively wide emission spectra of organic dyes and the overlap of their spectra near the low energy region. Few low molecular weight dyes have a combination of a large Stokes shift, which is defined as the separation of the absorption and emission maxima, and high fluorescence output. In addition, low molecular weight dyes may be impractical for some applications because they do not provide a strong enough fluorescent signal. Furthermore, the differences in the chemical properties of standard organic fluorescent dyes make multiple, parallel assays quite impractical since different chemical reactions may be involved for each dye used in the variety of applications of fluorescent labels.

Practical aspects of bioconjugation of thiol-stabilized CdTe nanoparticles with complementary antigen and antibody have been reported in the literature (Wand et al, 2002). However the bioactivity of the prepared immunocomplexes in this case was limited. Moreover, the size of nanoparticles was not precisely controlled. The possibility of the lymph node mapping was demonstrated by Kim et al (2004) using CdTe/CdSe core-shell nanocrystals. However, the use of these nanocrystals is restricted to applications where there is not significant absorption of infrared emission by biological tissue. An additional problem is the toxicity of such a composite, which limits the possible applications. The use of CdSe/ZnS nanocrystals as fluorescent labels for multiphoton microscopy was recently demonstrated by Larson et al (2003). Although the authors visualized quantum dots dynamically through the skin of living mice, this method is of limited usefulness because high pumping intensity is a critical requirement to achieve efficient multiphoton assisted excitation of nanocrystal luminescence. A direct method for conjugating protein molecules to luminescent CdSe—ZnS core-shell nanocrystals was described by Mattoussi et al (2000) and later by Goldman et al (2002). These bioconjugates have been proposed as bioactive fluorescent probes in sensing, imaging, immunoassay and other diagnostic applications. However, the bioconjugates are of relatively large size (30-45 nm in diameter) and had a quite limited solubility in water. As result these nanocomposites have only limited capability to penetrate through the cell membrane and can not be used very effectively for intracellular diagnostics. Also, water-soluble CdTe, CdₓHg₍₁₋ₓ₎Te and HgTe nanocrystals have been proposed for biolabeling of biocompatible polymers. In this work the nanocrystals were encapsulated into the polymer with the formation of microcapsules, which have been suggested as potential materials for monitoring the drug delivery process (Gaponik et al, 2003). Although the initial CdTe or HgTe nanocrystals demonstrated good water solubility and were of small size (46 nm) the final composites with the biopolymer were of several micron sizes and were too large to be used for intracellular drug delivery and diagnostics.

The invention is directed towards solving at least some of the problems with known systems.

### STATEMENTS OF THE INVENTION

According to the invention there is provided a method for quantitatively and qualitatively determining the presence of a macromolecule comprising the steps of:—
providing nanoparticles in a buffered solution:
adding a test sample to the buffered nanoparticle solution; and
measuring the difference between the buffered nanoparticles in the presence and absence of the test sample.

In one embodiment of the invention the nanoparticles are less than 100 nm in size.

In one embodiment of the invention the buffered solution comprises an inorganic buffer solution.

In another embodiment the buffered solution comprises a phosphate-based buffer solution.

In another embodiment the buffered solution comprises a tris-borate based buffer solution.

In one embodiment of the invention the buffered solution is prepared in water.

The macromolecule may be a protein, glycoprotein a peptide.

In one embodiment of the invention the difference between the buffered nanoparticles in the presence and absence of the test sample is measured by fluorescence.

In one embodiment of the invention the difference between the buffered nanoparticles in the presence and absence of the test sample is measured by fluorescence intensity and fluorescence life time imaging (FLIM).

In another embodiment the difference between the buffered nanoparticles in the presence add absence of the test sample is measured by estimation of the turbidity of the solution containing the nanoparticles.

The test sample may be selected from any one or more of blood, sputum, urine, ravage fluid, biopsy material, tissue sample, cultured or primary isolated cells.

In one embodiment of the invention the nanoparticles comprise a chemically attached entity.

In another embodiment the nanoparticles comprise an entity which has been chemically modified.

The invention also provides a method for promoting electrical stimulation or conductivity comprising:—
providing nanoparticles in the form of nano-wires;
adding a target compound;
applying a conductive force; and
measuring the difference in conductivity in the presence or absence of the target compound.

In one embodiment of the invention the nanoparticles are less than 20 nm in size.

The difference in conductivity may be measured using fluorescence imaging.

The invention provides a method for identifying a target compound useful in the preparation of a medicament for the treatment and/or prophylaxis of a disease state which involves a loss or change in electrical conductivity.

The disease state is selected from any one or more of spinal cord injuries, neuron and nerve damage, multiple sclerosis or any other neurodegenerative disease.

The invention further provides a method for determining intracellular transport and functional response in a cell comprising the steps of:—
applying nanoparticles to a cell type; and
measuring the fluorescence of the cells to determine the uptake and cellular distribution of the nanoparticles in the cell.

In one embodiment of the invention the nanoparticles are less than 20 nm in size.

In another embodiment of the invention the nanoparticle is associated with a biologically active entity.

In a further embodiment of the invention the nanoparticle comprises a chemically attached entity.

In one embodiment of the invention the method discriminates between the cytosolic and nuclear compartments of a cell.

Preferably the nanoparticles are up to 20=m in size. The nanoparticles may be up to 10 nm in size up to 5 nm in size or up to 3 nm in size.

In one embodiment of the invention the nanoparticles are water soluble.

In another embodiment the nanoparticles are lipid soluble.

In one embodiment of the invention the nanoparticles comprises II-VI colloidal nanoparticles.

In one embodiment of the invention the nanoparticles are CdTe nanoparticles. In another embodiment the nanoparticles are CdSe nanoparticles.

### BRIEF DESCRIPTION OF THE FIGURES

The invention will be more clearly understood from the following description thereof given by way of example only with reference to the accompanying figures in which:—

FIG. 1 is an example of the Cellomics Kineticscan View screen showing Nuclear to Cytoplasmic fluorescence intensity ratio in AGS cells accumulating CdTe QDs (CircRingAvgIntenRatio) in the Compartmental Analysis Bioapplication. Upper window, Nuc/Cyt intensity ratio in each individual cell in the well; middle panel, fluorescence detected in blue, green, red channels and composite image (left to right). Lower panel, numerical data from cells and outlined nuclear and cytoplasmic areas of the cell included in analysis.

FIG. 2 is a graph showing the Nuc/Cyt fluorescence distribution in fixed and fixed/permeabilised cultured epithelial cells (small dashed and large dashed lines, respectively). QDs size is increasing from left to right (experimental points 2-6). (FIG. 2A). FIGS. 2B and 2C show the intracellular fluorescence of green emitting QDs in permeabilised (B) and non-permeabilised cells (C).

FIG. 3 is a 96-well plate containing solutions of CdTe nanocrystals in water, PBS, PBS without Ca and Mg ions or culture medium with different amounts of bovine serum albumin (BSA) protein. The protein concentration of A is 0 mg/ml BSA, B is 2 mg/ml, C is 1 mg/ml, D id 0.5 mg/ml, E is 0.1 g/ml, F is 0.05 mg/ml and G is 0.01 mg/ml. FIG. 3A shows the 96-well plate illuminated with light illumination and FIG. 3B shows the 96-well plate with UV lamp illumination.

FIG. 4 shows the PL intensity decays of a solution of CdTe in water with two different amount of BSA, 0 mg/ml (a) and 2 mg/ml (B). Results of three-exponential analysis of decay curves are shown by the thick black lines with corresponding residuals (b) and (c). Insets show images of luminescence lifetime distribution obtained by FLIM technique scanning the sample across the square of 80×80 μm size.

FIG. 5 shows the PL intensity decays of a solution of CdTe in PBS with two different amount of BSA, 0 mg/ml (a) and 2 mg/ml (B). Results of three-exponential analysis of decay curves are shown by the thick black lines with corresponding residuals (b) and (c). The insets are as described in FIG. 4.

FIG. 6 shows the PL intensity decays of a solution of CdTe in PBS-without Ca and Mg ions, with two different amount of BSA, 0 mg/ml (a) and 2 mg/ml (B). Results of three-exponential analysis of decay curves are shown by the thick black lines with corresponding residuals (b) and (c). The insets are as described in FIG. 4.

FIG. 7 shows the PL intensity decays of a solution of CdTe in medium with two different amounts of BSA, 0 mg/ml (a) and 2 mg/ml (B). Results of three-exponential analysis of decay curves are shown by the thick black lines with corresponding residuals (b) and (c). The insets are as described in FIG. 4.

FIG. 8 is a bar chart showing the dependence of integrated PL intensity (upper panels) and values of averaged lifetimes τₐᵥ (bottom panels) on concentration of BSA protein. The CdTe are in water.

FIG. 9 is a bar chart showing the dependence of integrated PL intensity (upper panels) and values of averaged lifetimes τₐᵥ (bottom panels) on concentration of BSA protein. The CdTe are in PBS.

FIG. 10 is a bar chart showing the dependence of integrated PL intensity (upper panels) and values of averaged lifetimes τₐᵥ (bottom panels) on concentration of BSA protein. The CdTe are in PBS without Ca and Mg ions.

FIG. 11 is a bar chart showing the dependence of integrated PL intensity (upper panels) and values of averaged lifetimes τₐᵥ (bottom panels) on concentration of BSA protein. The CdTe are in medium.

FIG. 12 shows the synthesis of NPX-PEG-NH₂;

FIG. 13 shows an agarose gel electrophoresis of TGA QD's (sb 105-2(1)) mixed with EDC (lane 2) and increasing amounts of EDC and a 2 fold excess of NPX-PEG-NH₂ (lane 3, 4 and 5). UV filter at 516 nm and 12 s exposure time;

FIG. 14 is a FLIM lifetime image of cells;

FIGS. 15a and 15b are PL lifetime histograms obtained from regions A(a) and B(b) of FIG. 1 respectively;

FIG. 16 are images of a compartmental analysis;

FIG. 17 are fluorescent intensities registered by high content screening method in TBP-1 cells fixed with para-formaldehyde (PFA) permebilised with TritonX 100 and exposed to QDs of increasing sizes.

FIG. 18 are fluorescent intensities registered by high content screening method in Hep2 cell line fixed with para-formaldehyde (PFA) permebilised with TritonX 100 and exposed to QDs of increasing sizes.

FIG. 19 illustrates cellular distribution in prefixed Hep2 cells;

FIG. 20 illustrates cellular distribution in prefixed TBP-1 cells;

FIG. 21 illustrates distribution of fluorescent intensities in glutaraldehyde THP-1 cells exposed to QDs with different surface charge.

FIG. 22 illustrates high power magnification images of TBP-1 of fixed with glutaraldehyde cells and exposed to QDs with 5% positive surface charge.

FIG. 23 illustrates background fluorescence levels in THP-1 cells after glutaraldehyde fixation registered in different emission channels.

FIG. 24 illustrates cellular distribution in live TBP-1 cells exposed to QDs with different charge.

FIG. 25 illustrates cellular distribution in live TBP-1 cells exposed to QDs with 5% positive charge.

FIG. 26a to 26d illustrates high power magnification images of live TBP-1 cells exposed to QDs with 5% positive (a), 100% negative (b), 50% negative (c) and 10% (d) positive charge.

FIG. 27 is an image of a dot blot illustrating CdTe QDs binding to BSA, DNA, RNA, purified histones and nuclear extract (A-E, respectively). 4 μl amounts of QDs were applied on the nitrocellulose membranes with pre-bound nucleic acids and proteins.

FIG. 28 is an image of a dot blot illustrating CdTe QDs binding to BSA, DNA, RNA, purified histones and nuclear extract (A-E, respectively). 8 μl amounts of QDs were applied on the nitrocellulose membranes with pre-bound nucleic acids and proteins.

FIGS. 29 to 34 are graphs illustrating the effect of varying protein concentration on quantum dots; and

FIG. 35 is a fluorescent lifetime decay curve of quantum dots in tris-borate.

### DETAILED DESCRIPTION OF THE INVENTION

### Definitions

Living cell (Cell)—refers to the self-replicating biological structure enclosed by an outer membrane and containing cytoplasm, organelles and nucleic acids (i.e. viruses, prokaryotic bacterial cells, protozoa and eukaryotic cells of higher species and multicellular organisms).

II-VI colloidal quantum dots—are semiconductor nanoparticles of II-VI compounds prepared as a colloidal solution with size-dependent optical and electronic properties.

Optical illuminators/emitters—any source of ultraviolet, visible or infrared light and combinations thereof.

Chemical or physical linking—bond via covalent, noncovalent, hydrophobic, hydrophilic, electrostatic, van der Waals, hydrogen bonding, magnetic or electromagnetic interactions.

The following abbreviations are used throughout the text:

RFU—Relative fluorescence units
FLIM—Fluorescent lifetime imaging
PBS—Phosphate buffer saline

### QD—Quantum dot

CdTe—Cadmium telluride
CdSe—Cadmium selenide
TGA—Thioglycolic acid
BSA—Bovine serum albumin
DNA—Deoxyribonucleic acid
RNA—Ribonucleic acid

We have found Quantum Dots (QDs) to be very useful in a number of applications including use as dyes for multi-colour intracellular contrasting imaging, fluorescent detector systems responding to changes in protein-rich environment and QDs ability to serve as building blocks for formation of complex lattices of two- and three-dimensional nature.

The QDs of the invention offer a method a quantification using an unlimited range of emission wavelengths. This ability has been exploited over a range of applications. The QDs used in the invention are as described in detail in PCT/IE2005/000047 the entire contents of which are herein incorporated by reference.

The invention will be more clearly understood by the following examples thereof.

### Synthesis of CdTe Nanoparticles

CdTe nanocrystals capped with thioglycolic acid used in the experiments were synthesized in aqueous medium as reported earlier (Gaponik et al, 2002). Briefly, demineralised aqueous solutions containing Cd(ClO₄)₂.6H₂O and a stabilizer (thioglycolic acid, TGA) at pH 11.8 were treated by H₂Te gas, which was generated by the reaction of AM₂Te₃ lumps with 0.5 M H₂SO₄ under nitrogen. The mixture of was then heated under reflux under open-air conditions. This method enabled us to prepare good quality CdTe nanocrystals with a narrow (<10%) size distribution. Variation of the temperature and the duration of the heating during the preparation of CdTe nanocrystals determines the final size of the nanocrystals and as a result the colour and luminescence maximum of the solution. Thus green (with photoluminescence maximum at 563 μm) CdTe nanoparticles were produced after 15 min of heating under reflux, while red (with photoluminescence maximum at 602 nm) CdTe colloid solution were produced after 24 hours of heating.

We have utilised water-soluble thioglycolic capped CdTe nanoparticles of varying sizes for selective nuclear and nucleolar localisation of green CdTe QDs and cytoplasmic compartmentalisation of red QDs, dependent on size and surface chemistry. The CdTe nanoparticles showed limited cytotoxicity and proved to be suitable for biological systems.

Other non limiting examples of nanoparticles which can be used in relation to the invention may comprise semi conductor nanoparticles.

II-VI semiconductor nanoparticles: ZnO, ZnS, ZnSe, ZnTe, CdS, CdSe, CdTe, HgS, HgSe, HgTe.

III-V semiconductor nanoparticles: AlN, AlP, AlAs, AlSb, GaN, GaP, GaAs, GaSb, InN, InP, InAs, InSb.

Group IV semiconductor nanoparticles: Si, Ge, Si₁₋ₓGeₓ

Other possible nanoparticles include SiO₂ (silica), any transition metal oxide (e.g. TiO₂, ZrO₂, HfO₂, MoO₂, Fe₂O₃, Fe₃O₄, CO₃O₄, ferrites), siloxane nanoparticles, dendrimers (dendritic polymers) and organic polymer nanoparticles.

Two aqueous colloidal solutions of CdTe nanocrystals of 2-5 nm mean size were used for studies in biological systems.

Fluorescence-emitting semiconductor nanocrystals (quantum dots, QDs) have currently become a target of intensive efforts of scientists worldwide as promising material permitting generation of multi-colour labels suitable for industrial and biomedical applications. For biomedical purposes in particular, the critical requirements which need to be met are water-solubility, biocompatibility and selective functionalisation of nanoparticles (addition of the desired chemical groups, peptides, proteins or complex molecules) enabling the adaptation of QDs for specific uses.

The fulfillment of the above requirements commonly involves creation of core/shell structures, selection of adequate stabilisers and incorporation of adaptor or linker molecules enabling QDs to be physically conjugated with the functionalising molecules of interest. This inevitably leads to the generation of structures typically in the order of 15-25 nm in diameter for the stabilised QDs per se or larger, depending on the type of molecules used for subsequent conjugation with QDs surface. Such structures, although possessing a number of valuable characteristics for specific utilization, have intrinsic limitations for penetration into compartments smaller than their physical size thereby precluding their use for targeting more spatially confined environments, e.g. intracellular organelles and nucleus. Small QDs as represented by CdTe nanoparticles (devoid of enlarging shell) possess several unique features making them usable for a variety of biomedical purposes. These include QDs application as dyes for multi-colour intracellular contrasting imaging, fluorescent detector systems responding to changes in protein-rich environment and QDs ability to serve as building blocks for formation of complex lattices of two- and three-dimensional nature. Conjugates of medicinal drugs with small non-shell coated nanoparticles can be utilised for improved targeted compound delivery into cells.

### ODs as Contrasting Intracellular Dyes

A significant number of biological assays designed to characterise intracellular transport and functional responses, such as for example, signalling cascades largely exploit molecular translocation events occurring both within the cytoplasm and between cytosol and the nucleus. Therefore there is a growing demand in research institutions and pharmaceutical companies worldwide for the availability of multi-colour photo-stable dyes discriminating between cytosolic and nuclear compartments. Contemporary systems of high content screening, in particular are capable of analysing multiplexed experimental setups utilizing a variety of fluorescence-emitting reporters. However, the choice of reliably performing dyes currently on the market is limited to organic dyes with a limited choice of emission wavelengths, e.g. green-emitting calcein and fluorescein derivatives for cytosolic visualisation, blue DAPI and Hoechst range and far-red-emitting DRAQ-5 for nuclear imaging. These either commonly overlap in their emission spectra with a multitude of other popular labels (Alexa, FITC, TRITC etc.) or represent DNA-binding agents emitting in a short-range spectrum (UV) causing significant bleaching of other labels in multi-colour systems and photodamage in live cell studies.

### Example 1

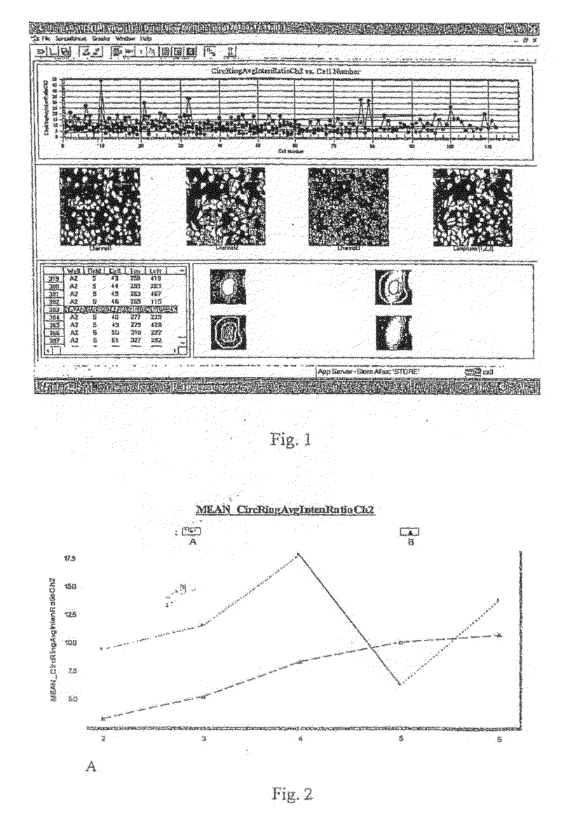

We applied a panel of CdTe quantum dots of different sizes to the routinely maintained macrophage-like cell line TBP-1 and epithelial cell line AGS in a 96-well plate format suitable for testing on the high content screening Cellomics® Kineticscan workstation. The system enables user-independent evaluation of the uptake and intracellular distribution of a large variety of fluorescent labels in the cells at individual and population level. The system performs an automated analysis of the registered events storing both the images of each individual cell and providing the fill quantitative analysis of the overall population dynamics. Similar systems are currently in use for carrying out large-scale screening of potential therapeutic compounds. A typical experimental acquisition screen of the Kineticscan View optimised for the work with QDs in cell systems is given on FIG. 1.

Experiments were carried out using fixed and fixed/permeabilised cells thus largely eliminating the influence of other QDs localisation factors apart from size selectivity, (such as pino- and phagocytic vesicle formation, nuclear pore activity and cytoskeleton-dependent transport mechanisms). FIG. 2 shows an example carried out in cultured epithelial cells. As seen from the FIG. 2(A), the average Nuc/Cyt fluorescence ratios were significantly higher in permeabilised cells (small dashed line) compared to non-permeabilised ones (large dashed line Δ) when the QDs of apparent particle size of less than 4 nm were used (points 2-4). The use of QDs with the estimated size of near 4 nm and over (emitting orange/red and red fluorescence) eliminated this difference (points 5 and 6 on the graph). These results demonstrate the possibility of utilization of CdTe QDs for a selective multi-colour dyes permitting to visualise discrete subcellular compartments.

### Example 2

### FLIM Data

In this case, Fluorescent Lifetime imaging method (SLIM) enables to detect interaction of QDs with target structures by registering changes in fluorescence emitting properties of QDs. Significant changes in fluorescence lifetimes may serve as an indicator of strong interaction of QDs with certain molecules or subcellular structures.

### Material and Methods

### Cell Lines

Fluorescence lifetime images were collected with the FLIM system (Microtime200 time-resolved confocal microscope system, PicoQuant) equipped with Olympus IX71 inverted microscope. The samples were excited by 480 nm picosecond pulses generated by a PicoQuant, LDH-480 laser head controlled by a PDL-800B driver. The setup was operated at a 20-MHz repetition rate with an overall time resolution of ˜150 psec. Lifetime maps were calculated on a pixel-by-pixel basis by fitting the lifetime of each pixel to the logarithm of the intensity and the FLIM system response was negligible compared with typical lifetimes of the quantum dots.

### Results

In the case when fluorophores are embedded in nonuniform environments, it has been shown that luminescence decays can be best understood by a model of continuous distributions of decay times. (Eftink 1991) Therefore, to gain a better insight into spatial distribution of lifetimes, the PL kinetics were evaluated from FLIM images:

maps of two-dimensional in-plane variations of the PL decay times measured in micro-PL setup. In this case every pixel in the lifetime image gives the lifetime at particular position in space (x,y).

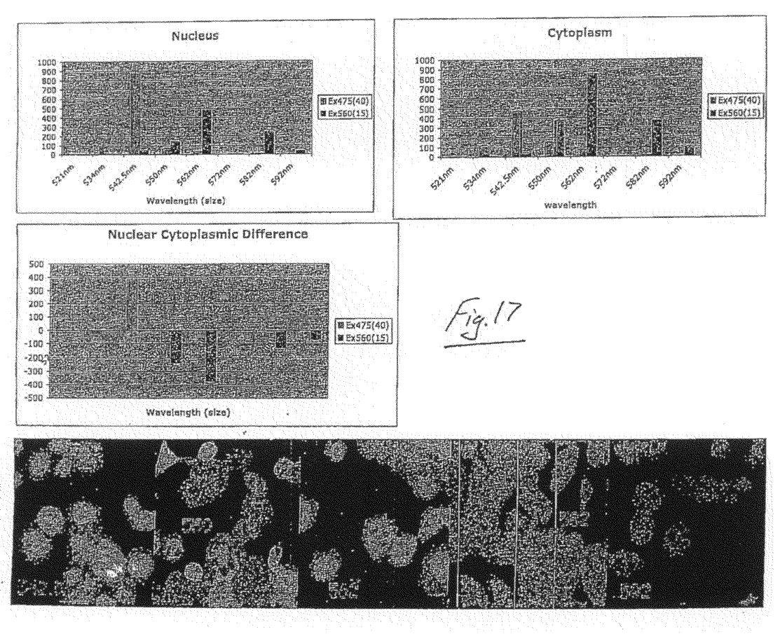

FIG. 14. Fluorescence lifetime image of cells. The image was collected at 300×300 pixel resolution with 4096 time channels; 2 ms acquisition time was provided per pixel and total recording time was 8.95 min. Image size: 27.2 μm×27.2 μm. Every pixel in the lifetime image (a) gives the lifetime at that particular position in space (x,y).

The lifetime image (FIG. 14) clearly demonstrates distribution of emitting species over cell cytoplasm, showing the longest PL decay time at the rim of cell (FIG. 14, region A) as compared to the region of nucleus (FIG. 14, region A).

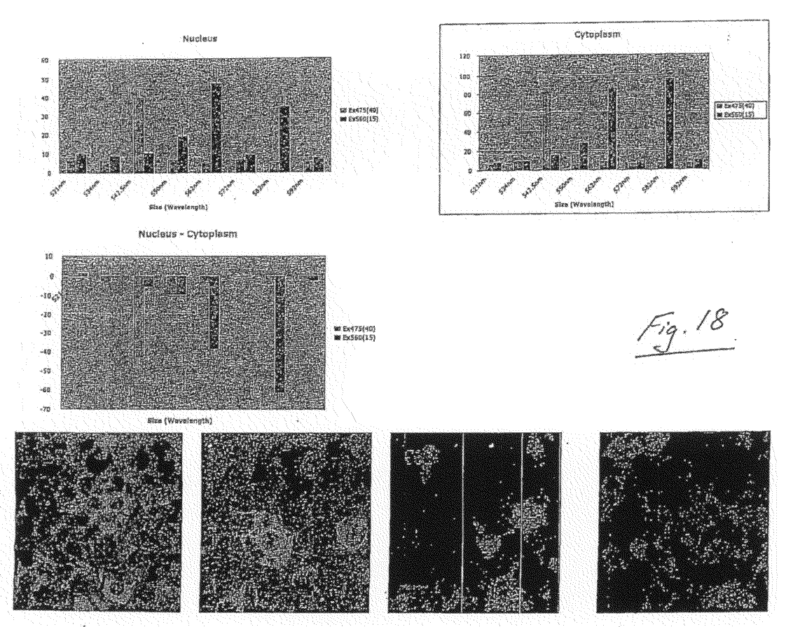

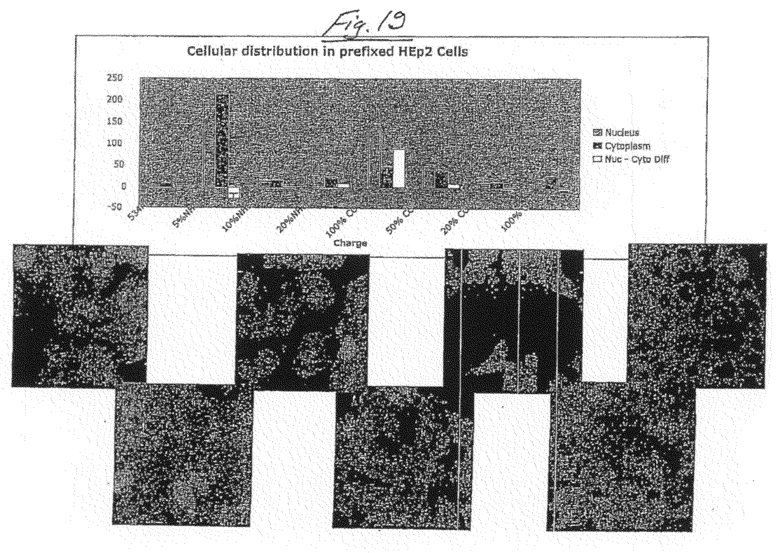

FIG. 15. PL lifetime histograms obtained from regions A (a) and B (b) of FIG. 14 respectively.

In both cases lifetime distributions consist of two maximums centered at 0.8 and 2.4 ns for region A and 0.8 and 4.5 ns in the region B. Comparing lifetime histogram obtained from different intracellular regions it is amply clear that drastic reduction of long-lived component is observed in region A Two-peak structure shown in FIG. 15

implies that at least two different decay processes are involved in nonradiative decay. The shorter lifetime can be attributed to the intrinsic recombination of initially populated electronic states in the core of quantum dots. (Bawendi, Carroll et al., 1992; Klimov, McBranch et al. 1999) Although origin of the longer component of PL lifetime is still in question, recent investigations strongly imply the involvement of surface states in the recombination process in colloidal quantum dots. (Wang, Qu et al. 2003). Faster decays observed in the region A implies that lifetimes of the emitting states are strongly reduced validating the presence of highly-efficient nonradiative energy transfer. In contrast, in region B the quenching-caused nonradiative pathways are no longer competing with the radiative pathways, resulting in 2-fold enhancement of lifetimes. It is noteworthy that intracellular accommodation of quantum dots in region A is accompanied by modification of only long-lived component, whereas the shorter component of PL lifetime is the same in both regions. This fact confirms strong involvement of surface states in the intracellular quenching mechanism.

### Quantum Dots for High Content Screening

The High Content screening and analysis systems enable to perform user-independent evaluation of the uptake and intracellular distribution of a large variety of fluorescent labels in the cells at individual and population level in multi-well format at a speed of up to several plates per hour. These systems perform an automated analysis of the registered events storing both the images of each individual cell and providing the full quantitative analysis of the overall population dynamics, including below-average responses. Appropriately designed fluorescent QDs with selective specificity and emission colour can be suitable for targeted visualisation of cellular organelles and multi-parametric analysis of cell population responses by means of high content analysis.

### Material and Methods

### Cell Lines

Two cell lines were used: HEp2 epithelial cell line, grown in minimum essential medium (Eagle) with Earles Salts, 10% Foetal Calf Serum, 2 mM L-glutamine; and Thp1 monocytic cell line (ECACC, Salisbury, England) grown in supplemented (10% foetal bovine serum; 2 mM/L L-glutamine; 100 μg/mL penicillin; 100 mg/mL streptomycin) RPMI 1640 media They were seeded out onto 96 well microtitre plates and onto coverslip slides at a concentration 2×10⁵ cells/nL. The HEp-2 cells were incubated for 24 hrs, and the Thp1 cells, cultured with 100 ng/mL PMA (to enable monocyte to macrophage differentiation), for 72 hours, both in controlled atmospheric conditions of 37° C., 5% CO₂.

Prefixed cells were washed twice in PBS, treated with 3% paraformaldehyde for 30 minutes, washed again and then permeabilised with 1.5% Triton X100 for 15 minutes. The plates were washed twice with PBS and 200 μL of PBS added. The plates were then sealed with parafilm and kept@ 4° C. until required. Cells were also prepared for live analyses as above; Thp1 cells seeded into a 96 well plate and HEp-2 cells into an 8 chamber coverslip slide (LabTech).

### Preparation of Quantum Dots

Two sets of quantum dots (QD) were used in this assay. One had a variation of size as measured in emission wavelength (521, 534, 542.5, 550, 562, 572, 582, and 592 nm). All these were CdSe/ZnS-DLcys with the exception of QDs 521 nm and 572 nm (CdTe—COO—). The other set (CdSe) were of the same size (534 nm) but varied in charge determined by the concentration of the conjugated amino group (5%, 10%, 20% NH3+) or carboxyl group (20%, 50%, 100% COO—) and hydroxyl group (100% OH). All dots were diluted to a concentration of 0.2 mg/mL in growth medium.

### Assay Protocols

### Prefixed Cells

The PBS was replaced by 100 μL of media, and 100 μL of diluted QDs were added. The cells were incubated for one hour, washed in media, stained with 1 μg/μL Hoescht for 3 minutes. washed with media and analysed using a Cellomics KineticScan®.

### Live Cells

Half of the media (100 μL/well from the 96 well plate; 200 μL/well from the 8 well plate) was replaced with the diluted QDs (charged particles only) and incubated for 1 to 3 hrs. the THP-1 cells were examined under the fluorescence microscope at 30 minutes, 1 hour and 3 hours. At 1 hour and at 3 hours, the cells were counterstained with Hoescht and fixed with 1% gluteraldehyde. This part of the experiment had to be repeated, with 3% paraformaldehyde used as a fixative instead of 1% gluteraldehyde.

### Analysis

The coverslips were examined using fluorescent and confocal microscopy. The images from the microtitre plates were acquired using the Cellomics KineticScan® and analysed later on the Cellomics Toolbox Scan® with the Compartmental Analysis® Bioapplications (CA). Using hoescht staining to identify the nucleus which is also defined as the object in Channel 1 (Ex 360(50); Em 515(20); blue), Compartmental Analysis can give information on the intensity of staining within the cytoplasm (Ring) and nucleus (Circ=Object) of the cells, as well as organelles within both the nucleus (CfrcSpot) and cytoplasm (RingSpot) in channels 2 (Ex 475(40); Em 515(20); green); and 3 (Ex 560(15); Em 600(25); red) (FIG. 16).

### Results

### Size

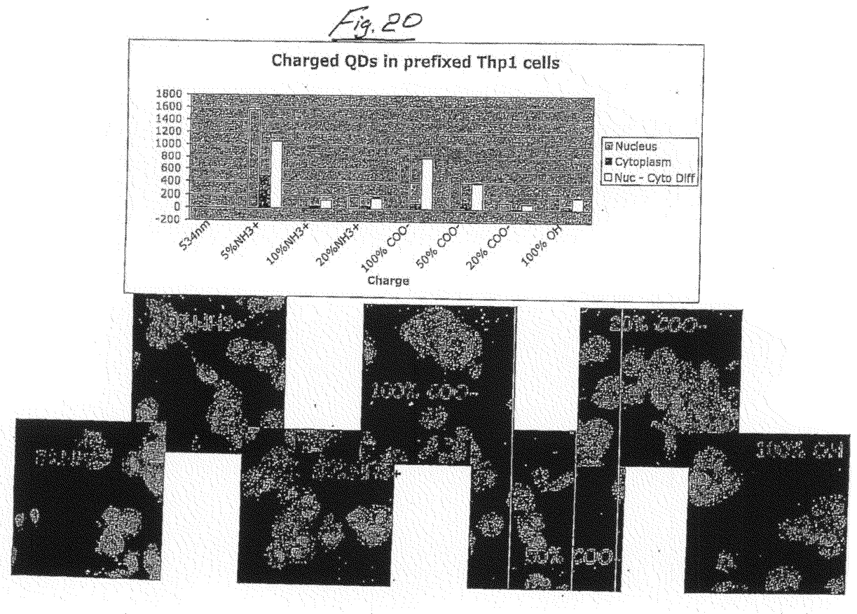

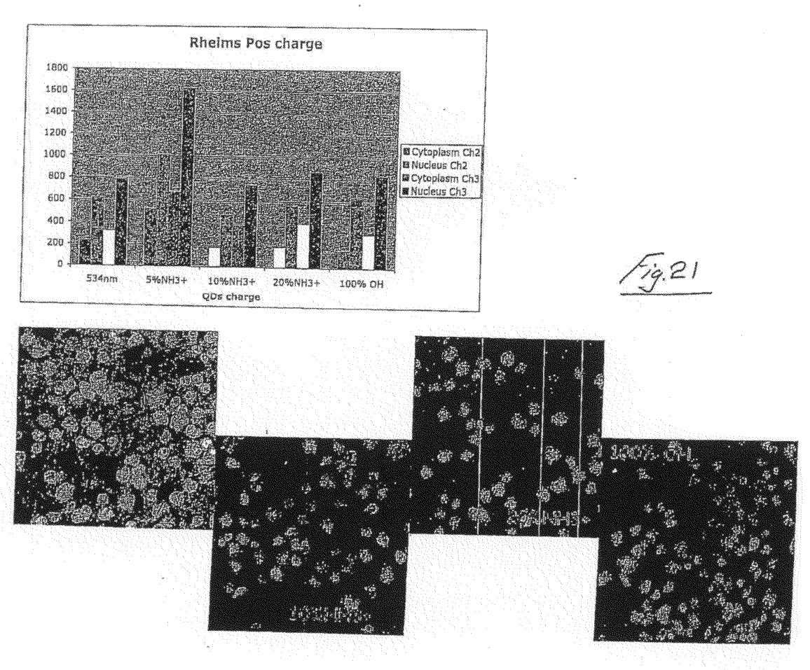

As described in previous studies, the size of the nanoparticle relates not only to where it locates within the cell but also at what wavelength it fluoresces. The particles added to cells that had been previously fixed in paraformaldehyde and permeabilised with TritoX100. The smaller sized particles went into the nuclei and emitted within the green wavelengths (λ542.5 nm), while the larger particles remained in the cytoplasm and emitted within the red wavelength (λ562 nm, λ572 nm, λ582 nm). The exception to these were the particles λ521 nm and λ572 nm, these showed no affinity for the cells at all. This was probably due to the modifications of these particular particles which also were negatively charged (FIG. 17 and FIG. 18). Of interest, the particle λ550 nm showed strong fluorescence in both channels but at different locations, the rim of the epitheliod cell line (HEp-2 cell) staining red while the cytoplasm stained green (FIG. 18).

### Charge

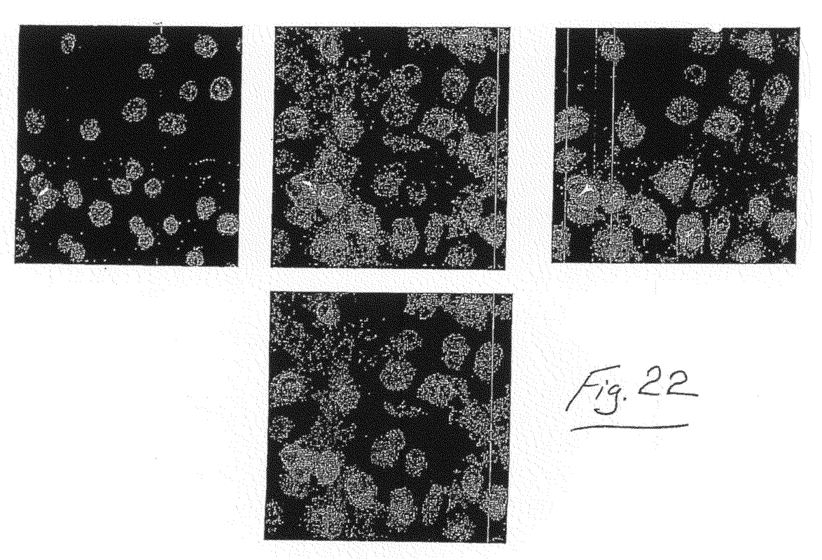

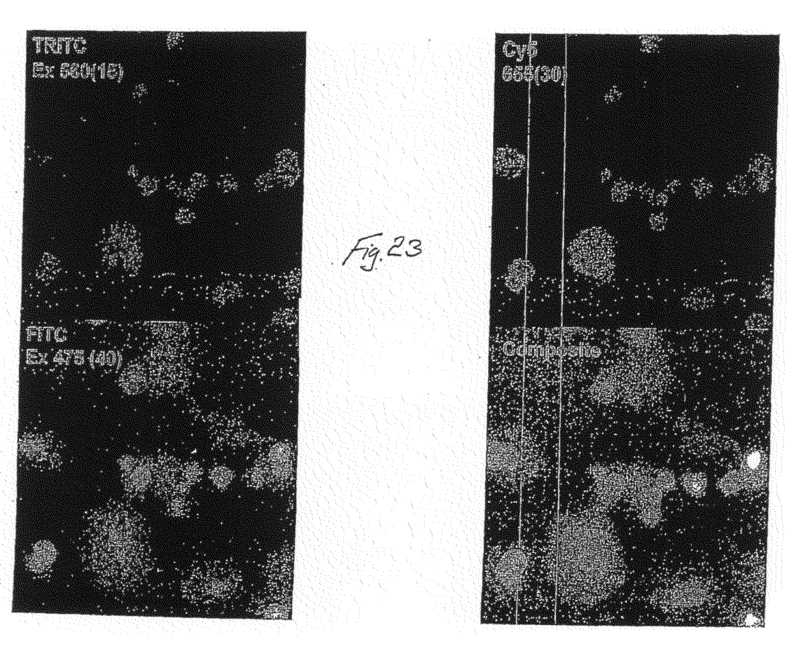

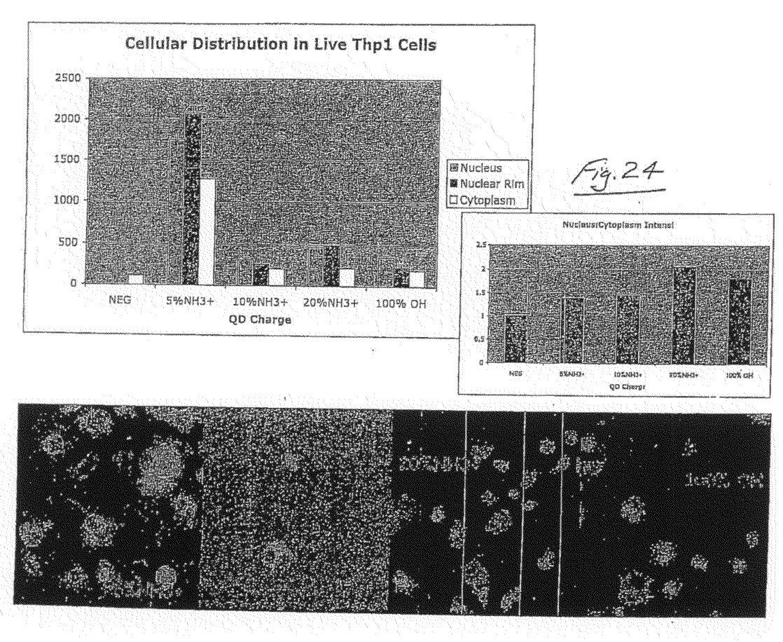

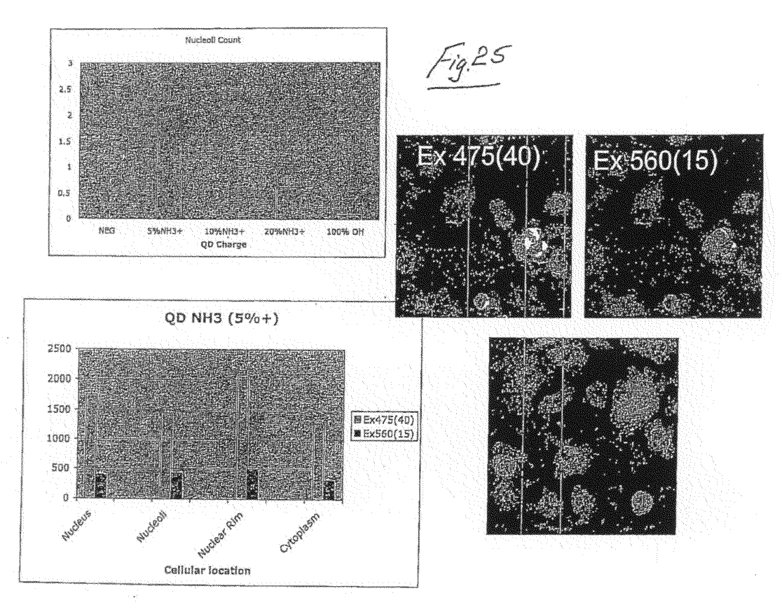

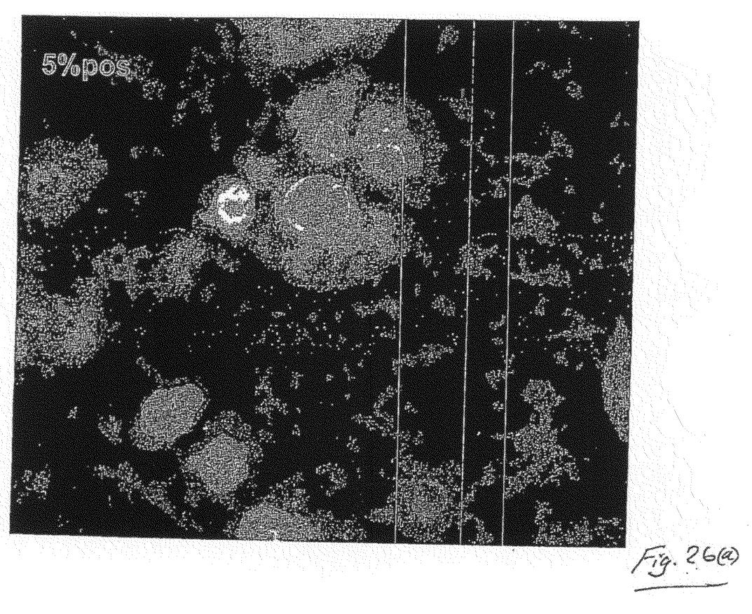

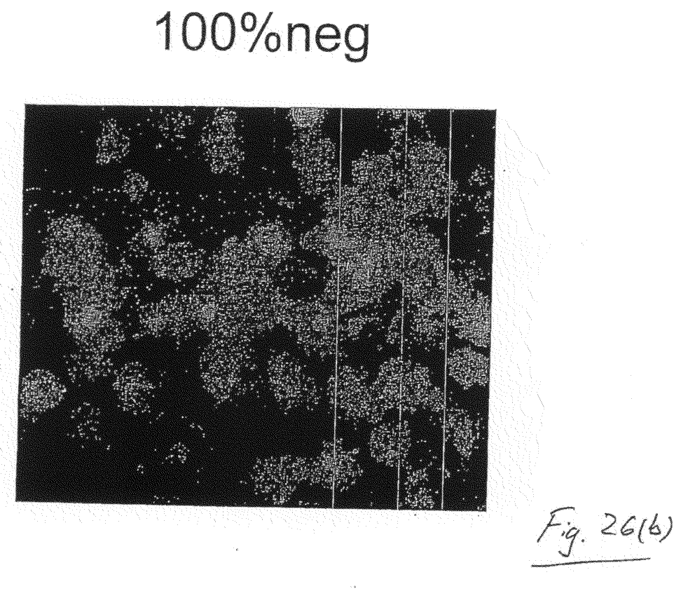

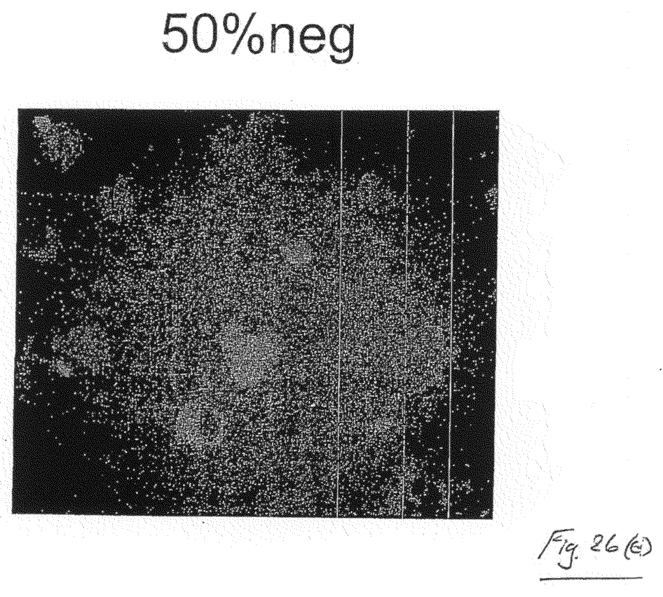

There were three negatively charged, three positively charge and one neutrally charged nanoparticles, which were tested with both prefixed and live cells. In the prefixed cells both TP-1 and HEp-2, all the particles tested positive in the green channel only. While in the Hep 2 cells the distribution was equally in the both the nucleus and the cytoplasm (FIG. 19), in the Thp1 cells, the nuclei picked up the dots more than the cytoplasm (FIG. 20). The 100% neg QDs located to the nucleoli in both cell lines. In the HEp-2 cells, the 5%+QDs appeared to have a more filamentous pattern, especially close to the nuclear rim. Examination of the QDs progress in the live cells was carried out using fluorescence microscopy (Nikon Eclipse TE 300) and on the UltraView Live Cell Imager confocal microscopy workstation (Perkin-Elmer Life Sciences, Warrington, England)(Nikon Eclipse TE 2000-U). In the HEp-2 cells, the 5%+QDs located onto what appeared to be the endoplasmic reticulum forming a meshwork surrounding the nucleus giving out fluorescence in the red spectrum. Apart from faint cytoplasmic fluorescence in the green channel with the 100%+QDs none of the other charged dots stained the HEp-2 cells. It was noted that even after 3 hours with the QDs the HEp-2 cells still seemed to be very healthy. The 5%+QDs stained the nucleoli. (red channel) in the Thp1 cells and also seem to accumulate at the nuclear rim (green channel). When examined under UV light there was also QDs in the cytoplasm. After fixing the THP-1 cells with 1% gluteraldehyde and counterstained with Hoescht, they were analysed on the Cellomics KineticScan. The 5%+QDs stained twice as strongly as the other dots in both channel 2 and channel 3. Interestingly, all the positively charged dots and the neutral QDs show fluoresecence in the nucleoli and at the nuclear rim (FIG. 21), especially the 5%+QDs (FIG. 22). No staining was noted with the negatively charged QDs. However it was noted that the negative control had some autofluorescence caused by fixing with gluteraldehyde (FIG. 23). Therefore, it was decided to repeat the assay using 3% paraformaldehyde as the fixative. While fixation with gluteraldehyde led to fluorescence in the red channel (FIG. 21), the green channel came to the fore with fixation with gluteraldehyde (FIG. 24) with the 5%+QDs being the only dots to fluoresce in both channels (FIG. 25). Unfortunately, the nucleoli appeared to stain in only a few cells with the 5%+QDs and the 20%+QDs. However, the nuclear rim stained quite strongly (FIG. 23). The negatively charged particles showed a weak speckled cytoplasmic pattern.

### CONCLUSIONS

We have confirmed that size of QDs affects where they locate within the cells. It is also important for the emitting wavelength. Conditions within the cells also affect the wavelength as can be seen when one part of the cell fluoresces green, yet another part fluoresces red. The QDs charge affects also how easily the cell will actively take up the QDs, the positively charged cells being more “appetising” than the negatively charged QDs. The positively charged QDs also seemed to be aiming for the nucleus, and getting into the nucleoli. Fixation is an important aspect of QD staining. We have shown that 1% gluteraldehyde enhanced the pattern already seen in the live cells.

### Quantum Dots for Protein Detection

Quantitative protein determination in complex solutions represents a routine task of most biochemical, immunological and general cell biology laboratories. To date, the choice of these methods is limited to traditional Bradford, Lowry methods or similar and their modifications, all of which are largely based of the formation of protein/reagent complexes providing a colorimetrically detectable reaction product. The readout is subsequently performed as light absorption measurement at a specific wavelength.

We have designed a system for protein quantification which exploits the specific destabilization of QDs solutions in the presence of physiological buffers. In the system, protein plays the role of a stabilising agent, maintaining QDs in fluorescence-emitting suspension. The higher the concentration of protein, the higher is the stability of the solution and hence the intensity of the fluorescent signal. The principle of quantitative stabilisation of QDs by protein solutions in the presence of opposite-acting destabilizing buffer holds true to the wide spectrum of CdTe quantum dots and therefore could be used in the fluorimetric systems working in a desired wavelength interval.

### Example 3

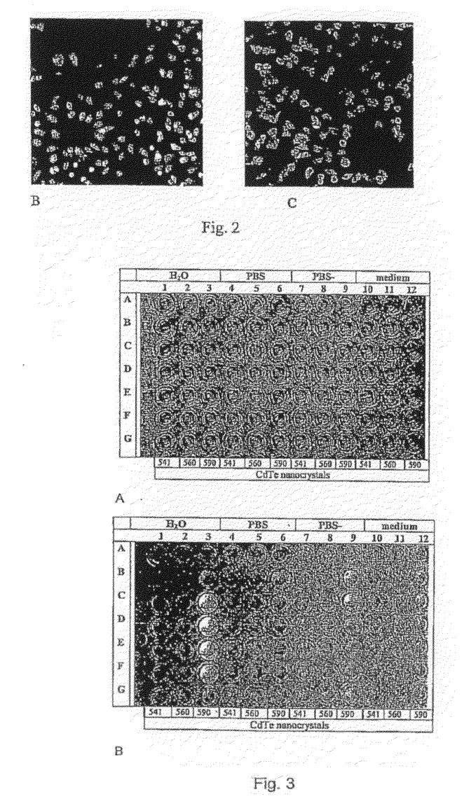

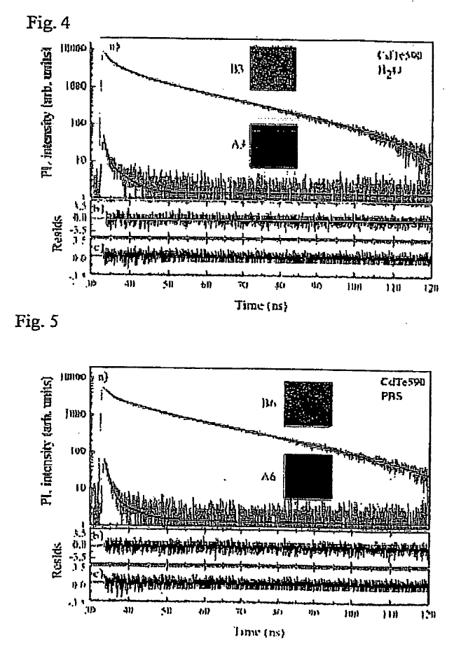

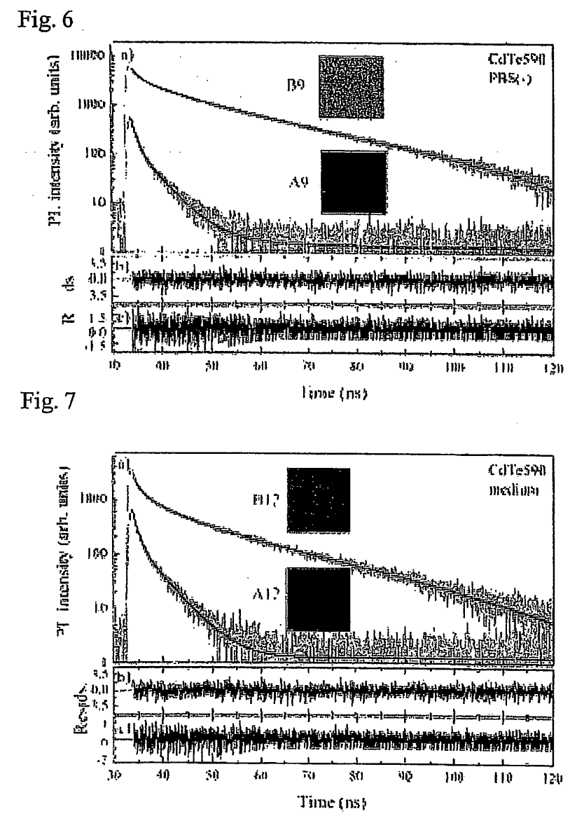

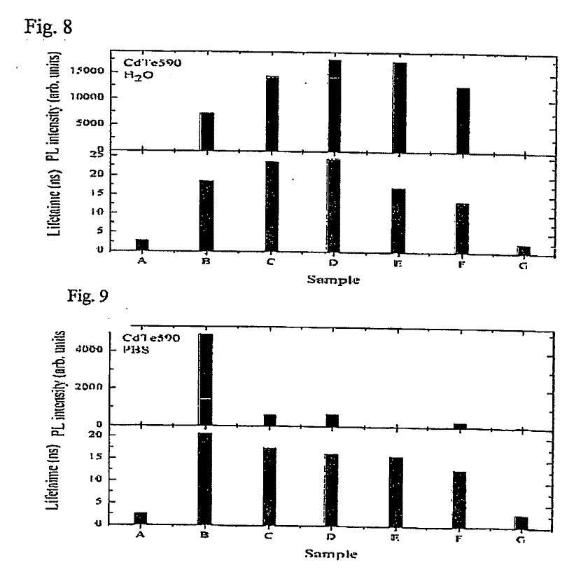

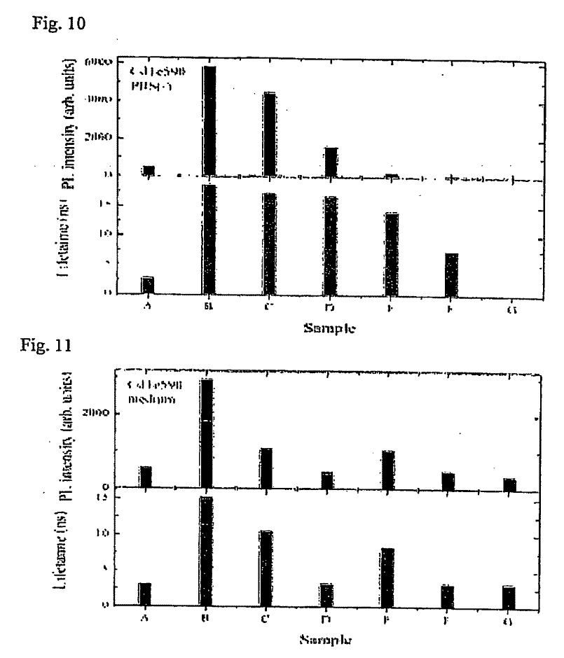

Three samples of CdTe QDs with distinctive fluorescence emission spectra (541, 560 and 590 nm, emitting closely to green, orange and red, respectively) were exposed to the increasing concentrations of purified bovine serum albumin solutions (0-0.01-0.05-0.1-0.5-1-2 mg/ml) in the presence of either de-ionised water, standard physiological phosphate buffer (PBS), PBS without Ca and Mg ions or routine culture medium (CO2-independent equivalent of medium RPMI 1640). Following a 20-min incubation at ambient temperature on a rotary shaker, the results of reaction were evaluated visually and using a spectroscopic methods. As seen from the FIGS. 3 (A-B), there is a detectable decrease of fluorescence intensity signal in the rows from B to G as a function of the decreasing protein concentration in the sample (2 to 0.01 mg/ml). Row A (containing no protein) yielded the poorest stability of the QDs solutions. This observation is further supported by time-dependent photoluminescence (PL) intensity decays of CdTe solutions (FIGS. 4 to 7) and by analysis of dependence of integrated PL intensity and values of averaged lifetimes on concentration of BSA protein (FIGS. 8 to 11).

Protein Concentration in the Wells (mg/ml)

| A | B | C | D | E | F | G |
|---|---|---|---|---|---|---|
| 0 | 2 | 1 | 0.5 | 0.1 | 0.05 | 0.01 |

PL decays were measured using time-correlated single photon counting (Time-Harp, Microtime 2000, Picoquant). The samples were excited by 480 nm picosecond pulses generated by Picoquant. LDH-480 laser head controlled by PDL-800B driver. The set-up was operated at a 20 MHz repetition rate with an overall time resolution of 150 psec. Decays were measured at 60000-80000 counts in the peak and reconvoluted using non-linear least squares analysis (FluoFit, PicoQuant) using an equation of the form:

I(τ)∝Σαᵢ exp (−t/τᵢ).

Where τᵢ are the PL decay times.

The pre-exponential factors αᵢ were taken into account by normalisation of the initial point in the decay to unity. The quality of fit was judged in terms of χ² value (with a criteria of less than 1.1 for an acceptable fit) and weighted residuals (FIG. 2-5 (b) (c)) The τᵢ and αᵢ parameters were used then to calculate the average Lifetime

$${τ}_{1} = \frac{\sum {α}_{1} {τ}_{i}^{2}}{\sum {α}_{1} {τ}_{i}}$$

The results indicate that there is a dose-dependent effect of BSA protein concentration in the sample on the stability and therefore light-emitting properties of QDs solutions. None of the existing methods of protein determination offers a practically unlimited range of emission wavelengths which can be utilized for this purpose.

The method may be used for the quantitative determination of other molecules possessing QDs-stabilizing properties in solutions using specifically chemically modified QDs. The method may also be used for quantitative evaluation of the presence of proteins with different properties using QDs with targeted chemical modifications.

### Example 4

### Proteins Data

We investigated the particular biopolymers to which the QD's are possibly binding/interacting with in the cell as the smaller green emitting QD's have been seen to go deep into the cell and have a distinctive sub-cellular distribution around the nucleoli 3. The main biopolymers associated with this region of the cell are DNA, RNA and histones, for this reason their interaction with the QD's is investigated. Each of these biopolymers was investigated separately.

### Experimental

### Materials

For the purposes of this study, green emitting cadmium telluride (CdTe) quantum dots capped with TGA were used. They are ˜2 nm in size and of a highly stable nature with a quantum yield of 15%. The DNA, RNA, and nuclear lysate used were extracted from Hut78 T-cells. Core histones were bought in from Medical Supply Company and the BSA was purchased from Sigma. The nitrocellulose used was purchased in from Millipore.

### Equipment

Fluorescent lifetime data was collected with the FLIM system (Microtime200 timeresolved confocal microscope system, PicoQuant) equipped with Olympus IX71 inverted microscope. The samples were excited by 480 nm picosecond pulses generated by a PicoQuant, LDH-480 laser head controlled by a PDL-800B driver. The setup was operated at a 20-MHz repetition rate with an overall time resolution of ˜150 psec. Plate read outs were carried out using the SPECTRAFluor Plus system (Tecan). There are a range of excitation (275 nm/360 nm/485 nm/590 nm) and emission (460 nm/465 nm/535 nm/595 nm) filters available. For the purpose of this research the 360 nm excitation and 595 nm emission filters were used. Ultra violet images were collected using a SONY transilluminator.

### Method

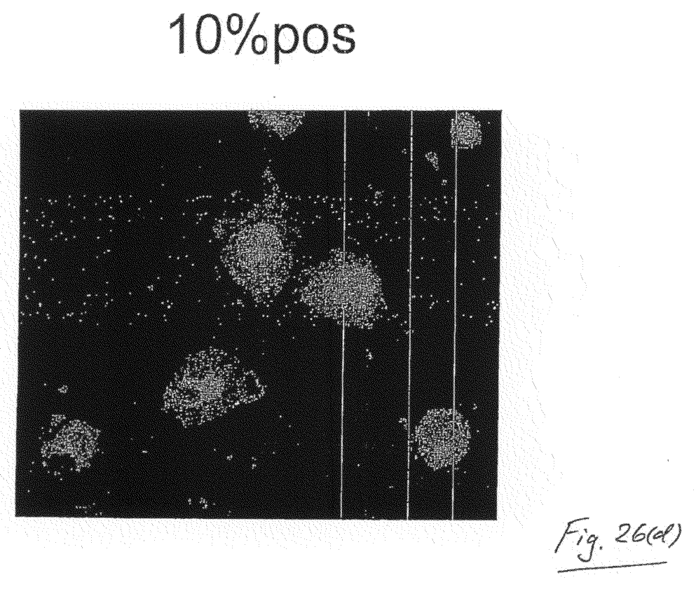

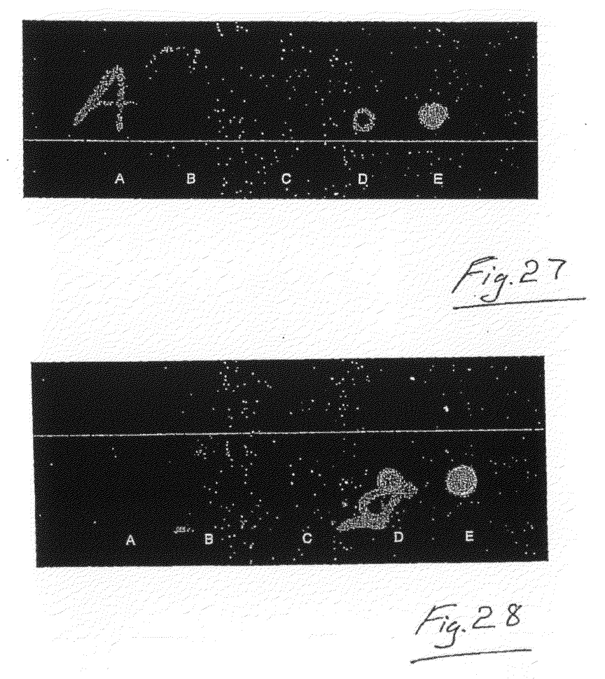

Nitrocellulose was used to bind the biopolymer samples, BSA, DNA, RNA, core histones and the nuclear lysate samples. Each sample was diluted to a concentration of 1 mg/ml using de-ionised water. 2 ug of each sample was then placed on the nitrocellulose (FIG. 27, FIG. 28). The quantum dots were used as received and diluted to one in a hundred using deionised water. The nitrocellulose was then “flooded” with the QD solution and incubated at 37° C. for ˜45 mins. The nitrocellulose was then washed rigorously twice using deionised water and kept moist at all times thereafter as the QD's deteriorate when they are allowed to dry out. The nitrocellulose was then imaged using the trans-illuminator.

### Results and Discussion

FLIM results

Table X below illustrates the different fluorescent lifetime decays obtained for the QD's when mixed with the core histones or DNA at various concentrations. For example at a concentration of 0.1 mg/ml, the QD's have lifetime 9.6 ns longer than that of the QD's in the histones.

TABLE X
Lifetime decay results for QD's mixed with core histones, DNA or RNA.
QD = CdTe TGA (30f, 19.05) Green emitting

| Core Histones + QD | | DNA + QD | | RNA + QD | |
|---|---|---|---|---|---|
| Conc. | Lifetime | Conc. | Lifetime | Conc. | Lifetime |
| (mg/ml) | Decay (ns) | (mg/ml) | Decay (ns) | (mg/ml) | Decay (ns) |
| — | — | 0.4 | 20.78 | — | — |
| 0.2 | 19.55 | 0.2 | 22.47 | 0.2 | 15.26 |
| 0.1 | 13.99 | 0.1 | 23.6 | 0.1 | 15.6 |
| 0.05 | 15.16 | 0.05 | 24.53 | 0.05 | 16.64 |
| 0.01 | 19.27 | 0.01 | 23.89 | 0.01 | 19.18 |
| 0.005 | 21.62 | 0.005 | 23.8 | 0.005 | 20.82 |
| 0.0001 | 20.11 | 0.0001 | 23.52 | 0.0001 | 14.18 |

There is a significant reduction in the lifetime of QD's with the histones as there is a reduction in concentration, however, there is no significant change in the lifetime of the QD's mixed with the DNA.

RNA also showed only to have an impact on the luminescence of the QD's at the very highest concentration, where a quenching effect was observed. [FLIM of whole cells shows a dramatic reduction in the lifetime of the QD's in the nucleus and nucleolus. The quenching effect of the RNA at high concentrations observed above may be a contributing factor.

### Plate Reader Results

Higher RFU of quantum dots mixed with core histones was observed when compared to that of QD's mixed with DNA.

TABLE Y
RFU results for QD's mixed with core histones or DNA.
CdTe TGA (30f, 19.05) Green emitting

| Core Histones + QD | | DNA + QD | |
|---|---|---|---|
| | Lifetime Decay | | Lifetime Decay |
| Conc. (mg/ml) | (ns) | Conc. (mg/ml) | (ns) |
| — | — | 0.4 | 1863 |
| 0.2 | 3837 | 0.2 | 1753 |
| 0.1 | 2649 | 0.1 | 1644 |
| 0.05 | 1460 | 0.05 | 1595 |
| 0.01 | 1304 | 0.01 | 1517 |
| 0.005 | 1520 | 0.005 | 1503 |
| 0.0001 | 1532 | 0.0001 | 1514 |

### Quantum Dot Binding Experiment

The whole methodology of using nitrocellulose to bind the biopolymers was used to establish whether or not the QD's would then bind to the biopolymers. No nonspecific binding of the QD's to the nitrocellulose was observed. FIG. 27 and FIG. 28 clearly illustrate the CdTe green emitting quantum dots bind to the histones. There is no binding to the BSA, DNA or RNA observed. A possible explanation for this could be attributed to the fact that the QD's are negatively charged and the histones are positively charged, thus there is an attractive force between them, whereas the DNA and RNA are negatively charged which results in a net negative force between them. This can be used for selective histone-mediated targeting of QDs to nuclei and nucleoli.

### Example 5

### Proteins and Buffers

Quantitative protein determination in complex solutions represents a routine task of most biochemical, immunological and general cell biology laboratories. To date, the choice of these methods is limited to traditional Bradford, Lowry methods or similar and their modifications, all of which are largely based of the formation of protein/reagent complexes providing a calorimetrically detectable reaction product. The readout is subsequently performed as light absorption measurement at a specific wavelength. We hereby suggest a system for protein quantification which is based on a different principle, exploiting specific destabilization of QDs solutions in the presence of physiological buffers. In this system, protein plays the role of a stabilising agent, maintaining QDs in fluorescence-emitting suspension. The higher the concentration of protein, the higher is the stability of the solution and hence the intensity of the fluorescent signal. The principle of quantitative stabilisation of QDs by protein solutions in the presence of opposite-acting destabilizing buffer holds true to the wide spectrum of CdTe quantum dots and therefore could be used in the fluorimetric systems working in a desired wavelength interval.

### Experimental

Fluorescence lifetime images were collected with the FLIM system (Microtime200 time-resolved confocal microscope system, PicoQuant) equipped with Olympus IX71 inverted microscope. The samples were excited by 480 nm picosecond pulses generated by a PicoQuant, LDH-480 laser head controlled by a PDL-800B driver. The setup was operated at a 20-MB repetition rate with an overall time resolution of ˜150 psec. Plate read outs were carried out using the SPECTRAFluor Plus system (Tecan). There are a range of excitation (275 nm/360 nm/485 nm/590 nm) and emission (460 nm/465 nm/535 nm/595 nm) filters available. For the purpose of this research the 360 nm excitation and 595 nm emission filters were used.

### Results and Discussion

The RFU and degree of polarisation of CdTe quantum dots was measured (Table Z), relative to the polarisation of QD's in water, using a Tecan Ultra evolution. Clearly the tris borate has a quenching effect on the QD's which would imply there are molecular interactions occurring, this is confirmed by the change in the degree of polarisation when compared to that of the QD's in water and other buffers.

TABLE Z
CdTe QD's mixed with various buffers ( 1/100 dilution).

| | RFU | | | Degree of polarisation | |
|---|---|---|---|---|---|
| PBS | 47142 | 33042 | PBS | 106.29 | 97.471 |
| ELISA | 41806 | 40347 | ELISA | 95.061 | 93.253 |
| HEPES | 30668 | 26834 | HEPES | 97.106 | 94.256 |
| TRIS | 35571 | 26216 | TRIS | 98.103 | 97.711 |
| Tris | 315 | 347 | Tris | 58.269 | 46.443 |
| Borate | | | Borate | | |
| H2O | 38111 | 30889 | H2O | 99.005 | 101.23 |
| Blank Well | 254 | 248 | Blank Well | 26.761 | 8.1313 |

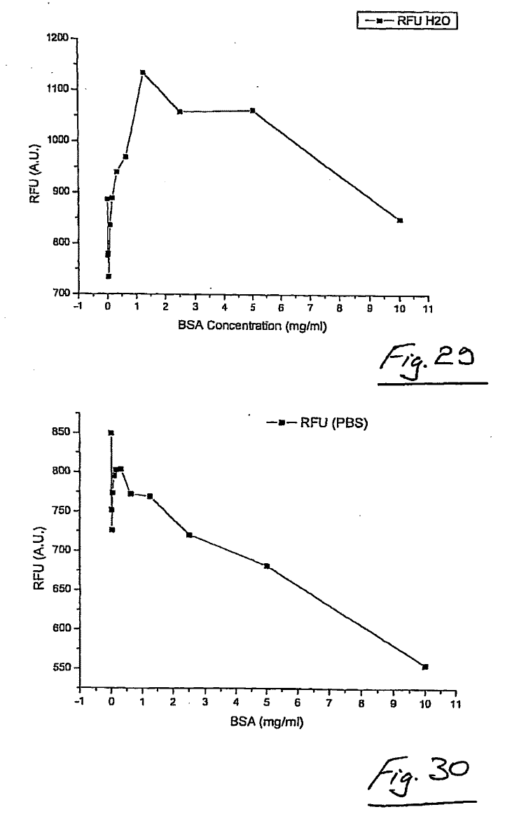

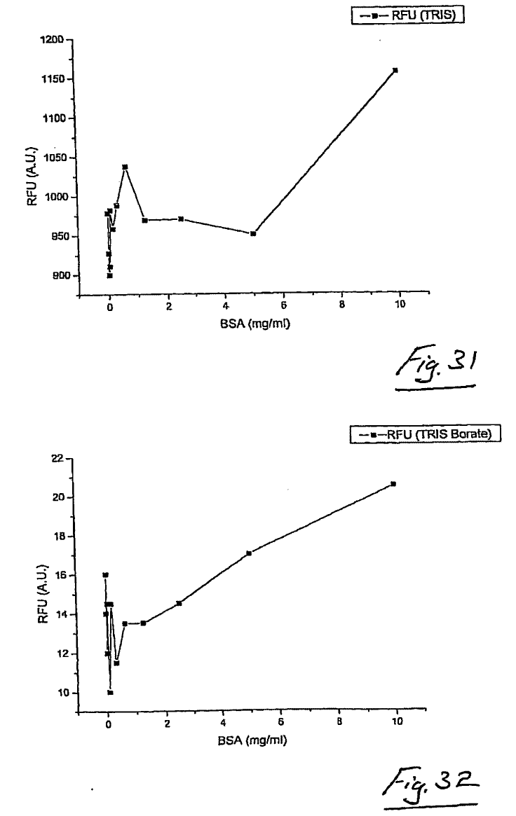

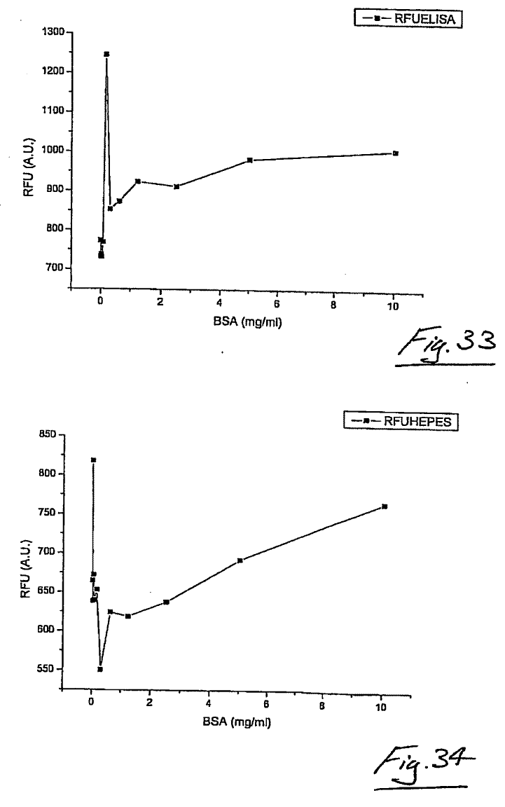

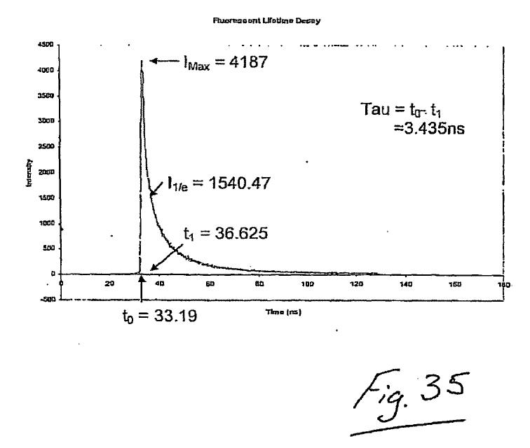

FIG. 29 to FIG. 34 show the effect that varying the protein concentration has on the QD's. In FIG. 31 to FIG. 34 there is an increase in the RFU observed for all of the buffers with the exception of the sharp peaks with ELISA and HEPES. This work is still under investigation and is to be repeated a number of times. From here it is expected to then concentrate on a particular buffer and protein concentration and vary the type of Qd's used. The fluorescent lifetime of the QD's is a measure of the average lifetime that the QD remains in an excited state before returning to the ground state. For the purpose of this research the fluorescent lifetime decay (τ1/e), was calculated using the following equations:(See FIG. 35 also).

τl/e=t1−t0

t0=t when intensity at max (Imax)
t1=t when intensity at l/e of max (Il/e)
where e=2.7 (natural log base)

### Il/e=Imax/e

The shortest lifetime of the QD is in tris borate (˜3 ns), hepes, tris and elisa share similar lifetimes of ˜15 ns, and PBS has a lifetime of ˜11 ns.

The effect of varying protein (bovine serum albumin (BSA)), concentration on the fluorescent lifetime of two different types of QD's are shown in Table S. It is clear to see that the decay times for both QD's reach a plateau at protein concentrations of 0.02 mg/ml to 0.005 mg/ml. This could be potentially due to QD concentration, which reaches saturation levels for given protein concentrations.

TABLE S
Fluorescent lifetime decays (ns) of two different quantum
dots (QD) with various protein concentrations

| | |
|---|---|
| QD (CdSe 592 nm) | QD (CdTe 593 nm) |

| | |
|---|---|
| Sample | Fluorescent lifetime decay [Tau (ns)] |

| | | |
|---|---|---|
| PBS | 6.0481 | 11.1946 |
| H2O | 6.7574 | NA |
| BSA 10 mg/mL | NA | 9.9632 |
| BSA 0.5 mg/mL | 5.6001 | 14.9635 |
| BSA 0.1 mg/mL | 3.4348 | 12.8364 |
| BSA 0.02 mg/mL | 6.7201 | 10.9333 |
| BSA 0.01 mg/mL | 6.9441 | 10.2244 |
| BSA 0.005 mg/mL | 5.1521 | 10.7841 |

### Multi Dimensional Signalling Networks

The nervous system in the human body is made up of billions of nerve cells, or neurons, organized in various networks. The majority of these neurons are located in the brain, brain stem and spinal cord, which constitute the so-called central nervous system (CNS). This network of interconnected neurons distributes messages as electrical impulses between the body and the brain. Messages that are received by the brain include sensory impulses that inform the brain about, for example, heat, pain or location of a part of the body. Conversely, messages are also sent by the brain to different parts of the body in order to elicit a muscle contraction that, for example, moves the hand from a burning flame.

Between adjacent neurons, there is a microscopic gap called the synaptic cleft. However small, the electrical signal carrying a message cannot bridge the synaptic cleft as it is. The solution to this is the synapse, an elegant way of bridging the gap chemically. The electrical impulse triggers the release of certain chemical substances into the gap. These substances are called neurotransmitters and are carried over the small synaptic cleft by diffusion. Once on the other side of the cleft, the neurotransmitters bind to certain proteins, called receptors, that are attached to the cell surface of the receiving cell. The binding of the transmitter to the receptor leads to the generation of a new electrical impulse.

The intensity and strength of the electrical impulse will decide which neurotransmitter to be released. Several medical disorders are caused by the dysfunction of neurotransmission in the central nervous system such as spinal cord injuries, neuron and nerve damage.

We found that CdTe particles of particular size were able to align/orientate themselves in a particular geometry permitting electrical stimulation and conductivity. We developed a technique to make CdTe nano-wires in physiological buffers (Volkov Y, et al)

Traditionally nano-wires are produced in cell-damaging toxic reagents. The ability to grow straight and branching nano-wires in a physiological solution is an advantage. Their use as conductors in this complex cell system using a network of nano-wires as a multi dimensional signalling structure may be of therapeutic value as electrical conductivity is a familiar feature for example, of multiple sclerosis.

We can grow nano-wires of different composition (QD size) to varying lengths in physiological buffers.

We found that nano-wires have an inherent ability to conduct electricity. Once a protein or a matrix or a firing neuron is present the ability to conduct along the wire is different. The conductivity of the nanowires are examined by patterning a surface with a matrix and then analysing the conductivity/fluorescence intensity along the wire (between two electrodes or measuring life time fluorescence imaging).

### Drug Conjugates

The compounds currently used in inflammation research and treatment (NPX-PEG-NH₂, Interferon α-2a) were used for conjugation with CdTe QDs and the efficiency of conjugation confirmed by biochemical methods as described below.

### Conjugates of CdTe-TGA Stabilised Quantum Dots.

TGA (tbioglycolic acid) stabilised quantum dots were prepared according to the published procedure (Gaponik 2002). The concentration of purified TGA-QD's solution were determined by mean of UV-absorption and PL emission as described in Yu, W. W. et al.

### Synthesis of Conjugates:

In a typical procedure, x mL of a purified TGA-QD's solution were dissolved in deionized water and mixed with x mL of an EDC (1-ethyl-3-(3-dimethylaminopropyl)-carbodiimide) solution in order to obtain a concentration ratio (R=QD's/EDC) from 2e⁻³ to 1. The reaction mixture is stirred at 0° C. or room temperature for 1 hour. Then, a desired amount of drug in solution (NPX-PEG-NH₂, Interferon α-2a) is added to the reaction mixture in accordance to the desired ratio (QD's)/(drug). The mixture is then stirred for 3 hours at RT.

### Purification:

After completion of the coupling reaction, precipitated formulations are purified by centrifugation and removal of supernatant. The operation is repeated until disappearance of free drugs in supernatant confirmed by Uv-vis absorption. The conjugates are suspended in a basic (pH=9) phosphate buffer.

Non-precipitated formulations are purified by gel exclusion chromatography over a G-25 column equilibrated in deionized water or phosphate buffer. All formulations are finally filtered over 0.2 μm filters.

### Characterisation:

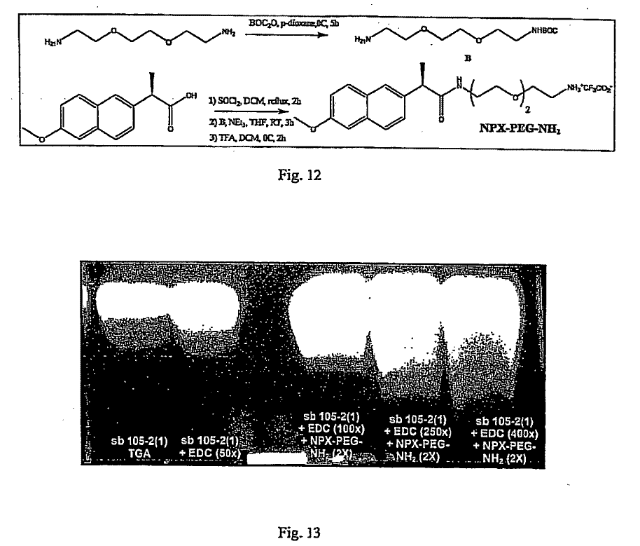

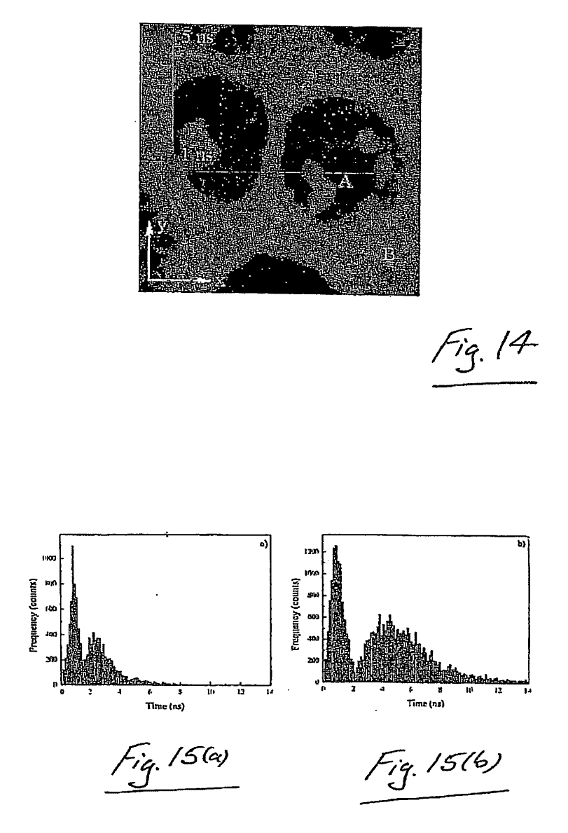

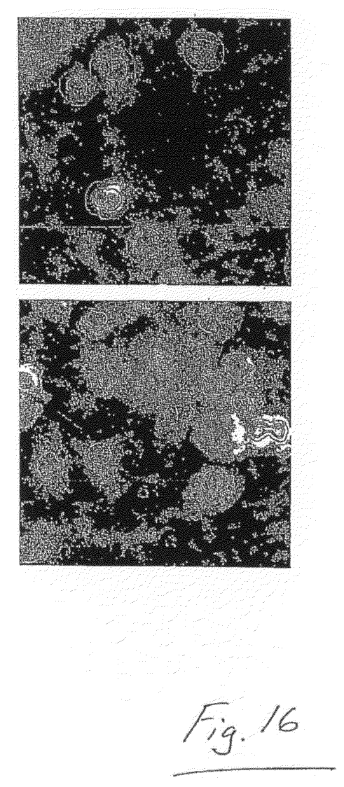

The drug coating on the nanoparticles is assayed by various techniques. UV-PL spectra of conjugates may show a shift in absorption or emission peak. The lifetime of the conjugated nanoparticles is compared with starting nanocrystal material. Finally, agarose gel electrophoresis experiment (FIG. 12 and FIG. 13) is performed. In a typical procedure, purified formulations (60-100 μL per well) are run in a 1% agarose gel in TRIS-HCl buffer (pH=8.1) for 1 h30, 76V-100 mA. Gels are revealed under UV lamp.

The invention is not limited to the embodiments hereinbefore described which may be varied in detail.

### REFERENCES

Akerman M E, Chan W C, Laakkonen P, Bhatia S N, Ruoslahti E Nanocrystal targeting in vivo. Proc. Natl. Acad. Sci. USA. 2002, 99, 12617.
Bruchez M Jr, Moronne M, Gin P, Weiss S, Alivisatos A P Semiconductor nanocrystals as fluorescent biological labels. Science. 1998, 281, 2013.
Chan W C, Nie S. Quantum dot bioconjugates for ultrasensitive nonisotopic detection. Science. 1998, 281, 2016.
Chan W C, Maxwell D J, Gao X, Bailey R E, Han M, Nie S. Luminescent quantum dots for multiplexed biological detection and imaging. Curr. Opin Biotechnol., 2002, 13, 40.
Dubertret B, Skourides P, Norris D J, Noireaux V, Brivanlou A H, Libchaber A In vivo imaging of quantum dots encapsulated in phospholipid micelles. Science. 2002, 298, 1759.
Gaponik N, Radtchenko I L, Gerstenberger M R, Fedutik Y A, Sukhorukov G B, Rogach A L. Labeling of biocompatible polymer microcapsules with near-infrared emitting nanocrystals. Nano Lett. 2003, 3, 369.
Gaponik, N.; Talapin, D. V.; Rogach, A. L.; Hoppe, K.; Shevchenko, E. V.; Kornowski, A.; Eychmuller, A.; Weller, H. Thiol-capping of CdTe nanocrystals: An alternative to organomettalic synthetic routes. Journal of Physical Chemistry B, 2002, 106, 7177-7185.
Goldman E R, Anderson G P, Tran P T, Mattoussi H, Charles P T and Mauro J M. Conjugation of luminescent quantum dots with antibodies using an engineered adaptor protein to provide new reagents for fluoroimmunosays, Anal. Chem. 2002, 74, 841.
Jaiswal J K, Mattoussi H, Mauro J M, Simon S M. Long-term multiple color imaging of live cells using quantum dot bioconjugates. Nature Biotechnol. 2003, 21, 47.
Kim S. Lim Y T, Soltesz E G, De Grand A M, Lee J, Nakayama A, Parker J A, Mihaljevic T, Laurence R G, Dor D M, Cohn L H, Bawendi M G and Frangioni J V. Near-infrared fluorescent type I quantum dots for sentinel lymph node mapping, Nature Biotechnology, 2004, 22, 93.

Larson D R, Zipfel W R, Williams R M, Clark S W, Bruchez M P, Wise F W, Webb W W. Water-soluble quantum dots for multiphoton fluorescence imaging in vivo. Science. 2003, 300, 1434.
Mattoussi H, Mauro J M, Goldman E R, Anderson G P, Sundar V C, Mikulec F V and Bawendi M G. Self-assembly of CdSe—ZnS quantum dot bioconjugates using an engineered recombinant protein, J Am Chem. Soc. 2000, 122, 12142.
Mattoussi H, Huno M K, Goldman E R, George P. Mauro J M. Optical Biosensors: Present and Future. Elsevier, The Netherlands 2002. (eds. Ligler F S and Rowe C A) pp 537-569.
Michalet, X., Pinaud, F., Lacoste, T. D., Dahan, M., Bruchez, M. P., Alivisatos, A. P., Weiss. S. Properties of semiconductor nanocrystals and their application to biological labelling. Single Molecules. 2001, 2, 261.
Peng Z A, Peng X Formation of high-quality CdTe, CdSe, and CdS nanocrystals using CdO as precursor. J Am. Chem. Soc. 2001, 123, 183.
Seydel C. Quantum dots get wet. Science. 2003, 300, 80.
Volkov Y, Mitchell S, Gaponik N, Rakovich Y P, Donegan J F, Kelleher D, Rogach A L. In-situ observation of nanowire growth from luminescent CdTe nanocrystals in a phosphate buffer solution. Chemphyschem. 2004 Oct. 18; 5(10):1600-2.
Wang S, Mamedova N, Kotov N and Chen W. Antigen/Antibody immunocomplex from CdTe nanoparticle bioconjugates, Nano Lett, 2002, 2, 817.
Yu, W. W.; Qu, L.; Guo, W.; Peng, X Experimental determination of the extinction coefficient of CdTe, CdSe and CdS nanocrystal. Chem. Mater., 2003, 15, 28542860.
Bawendi, M. G., P. J. Carroll, et al. (1992). “Luminescence properties of CdSe quantum crystallies: Resonance between interior and surface localized states.” J. Chem. Phys. 96(2): 946-954.
Dexter, D. L., T. Forster, et al. (1969). “The radiationless transfer of energy of electronic excitation between impurity molecules in crystals.” Phys. Stat Sol. 34(2): K159-162.
Eftink, M. R. (1991). Fluorescence quenching: Theory and application. Topics in Fluorescence Spectroscopy. J. R. Lakowicz New York, Plenum Press. 2.
Klimov, V. I., D. W. McBranch, et al. (1999). “Electron and hole relaxation pathways in semiconductor quantum dots.” Phys. Rev. B 60(19): 13740-9.
Wang, X., L. Qu, et al. (2003). “Surface-Related Emission in Highly Luminescent CdSe Quantum Dots.” Nano Lett. 3(8): 1103-1106.

1-34. (canceled)

35: A method for quantitatively and qualitatively determining the presence of a macromolecule comprising the steps of:—
providing nanoparticles in a buffered solution:
adding a test sample to the buffered nanoparticle solution; and
measuring the difference between the buffered nanoparticles in the presence and absence of the test sample.

36: The method as claimed in claim 35 wherein the nanoparticles are less than 100 nm in size.

37: The method as claimed in claim 35 wherein the buffered solution comprises an inorganic buffer solution.

38: The method as claimed in claim 35 wherein buffered solution comprises a phosphate-based buffer solution.

39: The method as claimed in claim 35 wherein buffered solution comprises a tris-borate based buffer solution.

40: The method as claimed in claim 35 wherein the buffered solution is prepared in water.

41: The method as claimed in claim 35 wherein the macromolecule is a protein.

42: The method as claimed in claim 35 wherein the macromolecule is a glycoprotein.

43: The method as claimed in claim 35 wherein the macromolecule is a peptide.

44: The method as claimed in claim 35 wherein the difference between the buffered nanoparticles in the presence and absence of the test sample is measured by fluorescence.

45: The method as claimed in claim 35 wherein the difference between the buffered nanoparticles in the presence and absence of the test sample is measured by fluorescence intensity and fluorescence life time imaging (FLIM).

46: The method as claimed in claim 35 wherein the difference between the buffered nanoparticles in the presence and absence of the test sample is measured by estimation of the turbidity of the solution containing the nanoparticles.

47: The method as claimed in claim 35 wherein the test sample is selected from any one or more of blood, sputum, urine, lavage fluid, biopsy material, tissue sample, cultured or primary isolated cells.

48: The method as claimed in claim 35 wherein the nanoparticles comprise a chemically attached entity.

49: The method as claimed in claim 35 wherein the nanoparticles comprise an entity which has been chemically modified.

50: A method for promoting electrical stimulation or conductivity comprising:—
providing nanoparticles in the form of nanowires;
adding a target compound;
applying a conductive force; and
measuring the difference in conductivity in the presence or absence of the target compound.

51: The method as claimed in claim 50 wherein the nanoparticles are less than 20 nm in size.

52: The method as claimed in claim 50 wherein the difference in conductivity is measured using fluorescence imaging.

53: The method as claimed in claim 50 for identifying a target compound useful in the preparation of a medicament for the treatment and/or prophylaxis of a disease state which involves a loss or change in electrical conductivity.

54: The method as claimed in claim 53 wherein the disease state is selected from any one or more of spinal cord injuries, neuron and nerve damage, multiple sclerosis or any other neurodegenerative disease.

55: A method for determining intracellular transport and functional response in a cell comprising the steps of:—
applying nanoparticles to a cell type; and
measuring the fluorescence of the cells to determine the uptake and cellular distribution of the nanoparticles in the cell.

56: The method as claimed in claim 55 wherein the nanoparticles are less than 20 nm in size.

57: The method as claimed in claim 55 wherein the nanoparticles is associated with a biologically active entity.

58: The method as claimed in claim 55 wherein the nanoparticles comprises a chemically attached entity.

59: The method as claimed in claim 55 for discriminating between the cytosolic and nuclear compartments of a cell.

60: The method as claimed in claim 35 wherein the nanoparticles are up to 20 nm in size.

61: The method as claimed in claim 35 wherein the nanoparticles are up to 10 nm in size.

62: The method as claimed in claim 35 wherein the nanoparticles are up to 5 nm in size.

63: The method as claimed in claim 35 wherein the nanoparticles are up to 3 nm in size.

64: The method as claimed in claim 35 wherein the nanoparticles are water soluble.

65: The method as claimed in claim 35 wherein the nanoparticles are lipid soluble.

66: The method as claimed in claim 35 wherein the nanoparticles comprises II-VI colloidal nanoparticles.

67: The method as claimed in claim 35 wherein the nanoparticles are CdTe nanoparticles.

68: The method as claimed in claim 35 wherein the nanoparticles are CdSe nanoparticles.
